(19) United States
(12) Patent Application Publication
(10) Pub. No.: US 2007/0293892 A1
(43) Pub. Date: Dec. 20, 2007

(54) SURGICAL THREAD FOR PLASTIC SURGERY AND METHOD OF IMPARTING TENSION TO SKIN

(76) Inventor: Katsuya TAKASU, Isshiki (JP)

Correspondence Address: KRATZ, QUINTOS & HANSON, LLP, 1420 K Street, N.W., Suite 400, WASHINGTON, DC, 20005, US

(21) Appl. No.: 11/467,215

(22) Filed: Aug. 25, 2006

(30) Foreign Application Priority Data
Jun. 16, 2006 (JP) 2006-167322

(51) Int. Cl. A61L 17/00

(52) U.S. Cl. 606/228

(57) ABSTRACT

A surgical thread for plastic surgery which is extremely simple in structure, is safe for human use, effectively removes sagging and wrinkling of skin, and further enables the tensing action to the skin to be maintained for a long period of time, that is, a surgical thread for plastic surgery comprised of a thread shaped member comprised of a thread body, to be implanted in the inside layers of the skin, partially formed in its longitudinal direction with projections for anchoring in the inside skin layers, wherein at least the thread body is comprised of an absorbable thread, and the thread body or the projections are formed with residual film parts which will not be absorbed by the inside skin layers, and a method of imparting tension to the skin using the same.

### BACKGROUND OF THE INVENTION

1. Field of the Invention

The present invention relates to a surgical thread for plastic surgery used for corrective surgery on the face and a method of imparting tension to the skin using the same.

2. Description of the Related Art

In the past, for example, for the purpose of erasing wrinkles from the face, the practice has been to inject collagen etc. into the deep parts of the wrinkles. The inventor previously proposed to extract collagen from the fat of the abdominal part of the human body for injection into the skin of the person extracted from and proposed a novel collagen harvesting system for effectively extracting fat from the human body and simply and efficiently separating the collagen from this extracted fat etc. (see Japanese Patent Publication B2) No. H6-11309). However, while injection of collagen is effectively in imparting pliability to the skin, it is not completely satisfactory in terms of eliminating sagging of skin.

Therefore, to counter sagging of the skin of the face, the surgical technique of “face lifts” for making an incision into the skin, separating the muscle over a broad range, lifting up and cutting away the excess subcutaneous tissue, then sewing closed the incision is known. This surgical technique eliminates sagging of the facial skin, but tends to cause a residual scar at the incision point, swelling of the face, or hemorrhaging under the skin for a relatively long time after the surgery.

Accordingly, in recent years, to erase wrinkles or eliminate sagging of the skin of the face more easily, the practice has been to use surgical thread for plastic surgery so as to impart a tensing action to the skin of the face. This method does not involve any major surgery. Further, the skin of the face is left with needle marks where the skin was punctured to pass the thread, but these needle marks disappear after a few days. Further, if using absorbable thread later absorbed in the body, no foreign matter is left in the body, so this practice is regarded as being particularly safe.

However, such surgical thread degrades along with time resulting in a drop in the skin tensing action. Further, when using surgical thread made of absorbable thread, the thread is absorbed by the body and disappears after about three months to one year. Therefore, the skin can be given a tensing action for only a limited time period.

### SUMMARY OF THE INVENTION

An object of the present invention is to provide a novel surgical thread for plastic surgery which is extremely simple in structure, is safe for human use, effectively eliminates sagging and erases wrinkles of skin, and further enables the tensing action to the skin to be maintained semipermanently, and a method of imparting tension to the skin using the same.

That is, according to a first aspect of the present invention, there is provided a surgical thread for plastic surgery comprised of a thread shaped member comprised of a thread body, to be implanted in the inside layers of the skin, partially formed in its longitudinal direction with projections for anchoring in the inside skin layers, wherein at least the thread body is comprised of an absorbable thread, and the thread body or the projections are formed with residual film parts which will not be absorbed by the inside skin layers. Due to this, sagging or wrinkles of the skin can be effectively eliminated or erased by an extremely simple structure. Further, a tensing action to the skin can be maintained for a long period of time. Further, since at least the thread body is formed by an absorbable thread, application to the human body is extremely safe.

Preferably, the projections are formed integrally with the thread body. Since projections are formed integrally with the thread body, projections can be formed extremely simply and effectively.

More preferably, the projections are thorn shaped members. Since the projections are thorn shaped members, they can be reliably anchored in the inside skin layers. Further, when implanting the surgical thread for plastic surgery in the inside skin layers, it is possible to keep the trauma to the skin and body tissue to a minimum.

Still more preferably, the residual film parts are formed at parts of the thread body other than at the projections. Since the residual film parts are formed at parts of the thread body other than the locations of the projections, the tension action to the skin given by the skin tissue latching onto the residual film parts can be maintained reliably over a long period.

Still more preferably, the residual film parts are comprised of gold. Since the residual film parts are comprised of gold, new skin tissue formation can be further promoted and the skin can be improved in pliability.

According to a second aspect of the invention, there is provided a method of imparting tension to skin comprising implanting any of the above surgical threads for plastic surgery into the inside layers of the skin, using the anchoring force of the projections of the surgical thread to impart a tensing action to the skin until the thread body comprised of the absorbable thread is absorbed, and using thread-like parts formed at the inside skin layers by skin tissue latching onto the residual film parts to impart a tensing action after the thread body is absorbed. This enables a tensing action to be given to the skin over a long period of time by an extremely simple configuration. Further, since the thread body is absorbed in the body, it is high in safety.

### BRIEF DESCRIPTION OF THE DRAWINGS

These and other objects and features of the present invention will become clearer from the following description of the preferred embodiments given with reference to the attached drawings, wherein:

FIG. 1 is a cross-sectional view of a surgical thread for plastic surgery according to an embodiment of the present invention;

FIG. 2 is a cross-sectional view along the line A-A of FIG. 1;

FIG. 3 is a schematic perspective view of the residual film part;

FIG. 4 is a cross-sectional view of a surgical thread for plastic surgery according to another embodiment;

FIG. 5 is a front view of a surgical thread for plastic surgery according to another embodiment;

FIG. 6 is a schematic view of the state of implanting in surgical thread for plastic surgery in the inside layers of the skin of the face;

FIG. 7 is a cross-sectional view showing the state where a surgical thread for plastic surgery is implanted in the inside layers of the skin;

FIG. 8 is a cross-sectional view showing the state of skin tissue latching onto the residual film parts; and

FIG. 9 is an enlarged a cross-sectional view showing the thread-like parts of skin tissue latching onto the residual film parts.

### DESCRIPTION OF THE PREFERRED EMBODIMENTS

Preferred embodiments of the present invention will be described in detail below while referring to the attached figures.

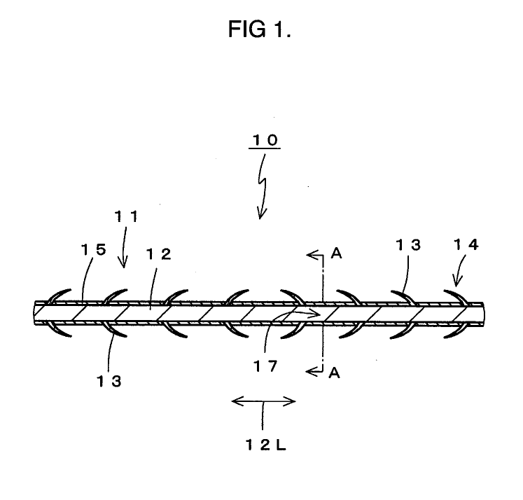

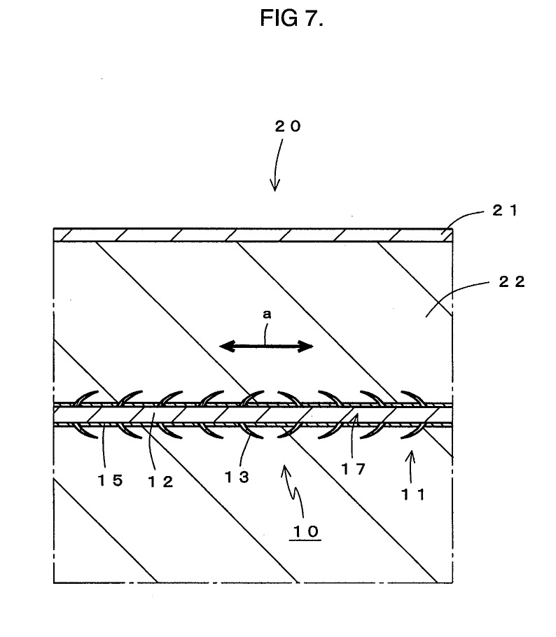

As shown in FIG. 1 and FIG. 7, the surgical thread for plastic surgery 10 is comprised of a thread shaped member 11 comprised of a thread body 12, to be implanted in the inside layers of the skin 22, partially formed in its longitudinal direction 12L with projections 13 for anchoring in the inside skin layers 22, wherein at least the thread body 12 is comprised of an absorbable thread, and the thread body 12 or the projections 13 are formed with residual film parts 15 which will not be absorbed by the inside skin layers 22.

The thread shaped member 11 is partially formed, along the longitudinal direction 12L of the thread body 12 to be implanted in the inside skin layers 22, with projections 13 for anchoring at the inside skin layers 22 (see FIG. 1 and FIG. 7). As the thread body 12, a conventional one known as surgical thread for plastic surgery is used. For example, the thread body 12 may be a bundle of a large number of fine filaments or may be formed from a single filament. Further, its diameter and length are not particularly limited, but as a specific example a diameter of about 0.01 to 1.0 mm or so and a length of about 10 to 60 cm may be mentioned.

The thread body 12 is comprised of absorbable thread. This absorbable thread breaks down and is absorbed in the human body along with the elapse of time. Further, even if the thread body 12 is not absorbed in the body and its original form does not completely disappear, as explained above, it is sufficient that thread-like parts be effectively formed by skin tissue latching on to the residual film parts 15. As the thread body 12, a polyglycolic acid (PGA), polylactic acid, polydioxanon (PDS), trimethylene carbonate (TMC), polyepsilon caprolactam, catgut, or other absorbable thread may be used. The time taken for such absorbable thread to break down and be absorbed in the body differs depending on the type of the absorbable thread, but, as explained later, generally three months to one year or so is preferable from the viewpoint of forming thread-like parts by the skin tissue by implanting the surgical thread for plastic surgery into the inside skin layers.

The thread body 12 is partially formed along its longitudinal direction 12L with projections 13. In the embodiments, in FIG. 1 and FIG. 7, the projections 13 are formed at equal intervals on the thread body 12 symmetrically in the vertical direction, but so long as they may be anchored at the inside skin layers 22, the projections arranged at the thread body 12 are not particularly limited in interval, direction, angle of inclination from the thread body, etc. For example, the projections may also be formed at only one side of the thread body, may also be formed alternately at one side and another of the thread body, and may also be formed arranged spirally around the circumference of the thread body.

The projections 13 are not particularly limited in material. They may be the above-mentioned absorbable thread or may be nylon, silk, stainless steel, polyester, polyethylene, polypropylene, or other nonabsorbable thread not breaking down and being absorbed in the body.

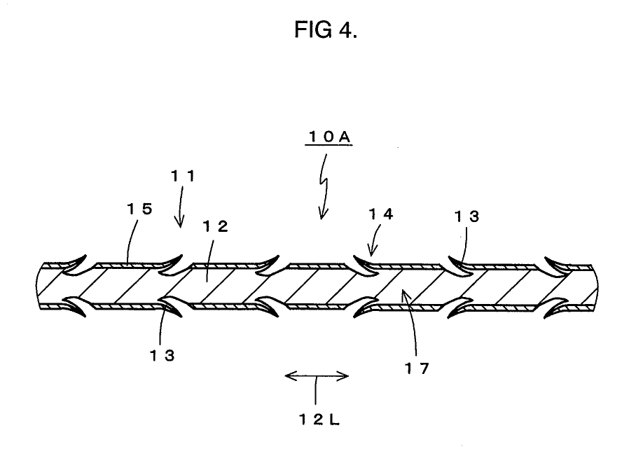

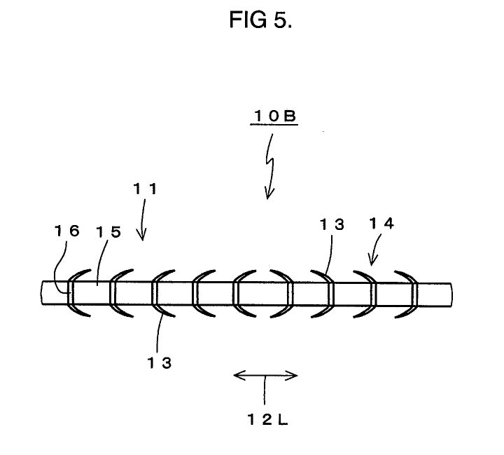

The projections 13 are formed on the thread body 12 by a conventional known technique. In another embodiment shown in FIG. 4, the thread body 12 of the surgical thread for plastic surgery 10A may be formed with cuts etc. by a known processing method so as to form the projections 13 integrally with the thread body 12. By adopting this configuration, the projections may be formed extremely simply and effectively. On the other hand, as in the embodiment of FIG. 5, the projections 13 of the surgical thread for plastic surgery 10B need not be formed integrally with the thread body 12, but may be provided with attachment parts 16 for attachment to the thread body 12. In this case, for example, it is possible to select different materials for the thread body 12 and projections 13 so as making only the thread body out of absorbable thread and making the projections out of nonabsorbable thread.

The projections 13 may be of any shapes such pins, knobs (not shown), etc. so long as they enable anchoring in the inside skin layers 22, but the projections 13 are preferably thorn shaped members 14 (see FIG. 1, FIG. 4, and FIG. 5). This is so that when the surgical thread for plastic surgery is implanted at the inside skin layers, the trauma given to the skin, the tissue, etc. due to the projections formed at the thread body can be greatly alleviated and more reliable anchoring at the inside skin layers becomes possible.

The residual film parts 15 are partially formed at the thread body 12 or the projections 13. As explained later, it is sufficient that after the thread body breaks down and is absorbed in the body, the skin tissue latches on the residual film parts at the inside skin layers to form thread-like parts. The residual film parts are not particularly limited in length and thickness. The residual film parts 15 have thicknesses of for example about 0.01 to 1000 μm or so.

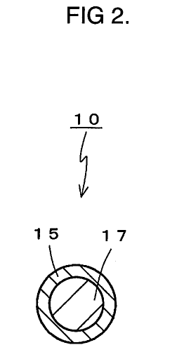

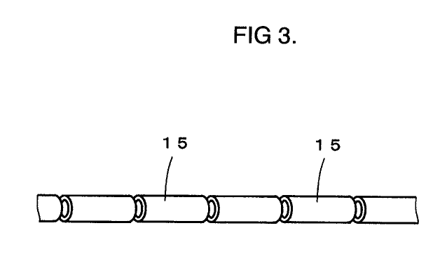

The residual film parts 15 are preferably formed at parts of the thread body 17 other than the projections (see FIG. 1, FIG. 4). At this time, as shown in FIG. 2, the residual film parts 15 are formed in a cylindrical shape so as to cover the surface of the thread body 17 other than the projections. Further, when the surgical thread for plastic surgery 10 is implanted in the inside skin layers, then the thread body is absorbed, as shown in FIG. 3, only the cylindrical residual film parts 15 remain in the inside skin layers. By forming the residual film parts in this way, the skin tissue newly created along with the absorption of the thread body in the inside skin layers reaches and latches on to the insides of the cylindrical residual film parts from their openings whereby the later explained thread-like parts are effectively formed. For this reason, the tensing action on the skin can be maintained more reliably for a long period of time.

As the method for partially forming the residual film parts 15 at the thread body 12 or the projections 13, the conventionally known plating, vacuum deposition, sputtering or other physical vapor deposition, chemical vapor deposition, etc. may be used. Specifically, cuts may be formed in the thread body formed with the residual film parts to form the projections (see FIG. 4) or, while not shown, the thread body 12 may be masked, then the residual film parts formed, then projections provided etc. so as to form the surgical thread for plastic surgery 10.

The residual film parts 15 are not particularly limited in material so long as they do not break down and are not absorbed in the body, but stainless steel, platinum, gold, or another metal, an inorganic compound, organic compound, etc. may be used. Among these, the residual film parts 15 are preferably made of gold. Note that the “gold” referred to here includes alloys comprised of gold and other metals.

Gold has the action of suppressing allergic reactions in the human body and also promises the synergistic effect of increasing the formation of collagen or other skin tissue at the inside skin layers. For this reason, by making the residual film parts from gold, not only are skin sagging and wrinkles eliminated and erased, but also new growth of skin tissue is promoted and the skin can be given bounce and pliability.

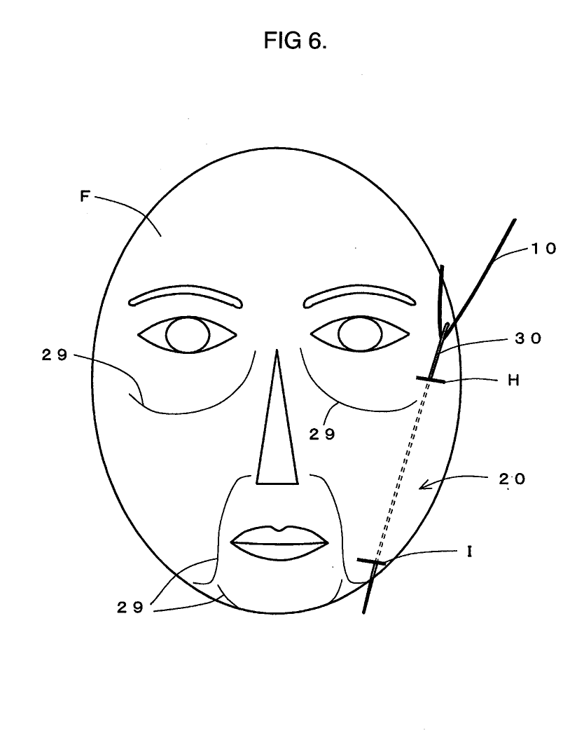

Next, the method of imparting tension to the skin using the surgical thread for plastic surgery 10 will be explained using FIG. 6 to FIG. 9. FIG. 6 shows an embodiment using the surgical thread for plastic surgery 10 to impart a tensing action to the skin 20 of the face F.

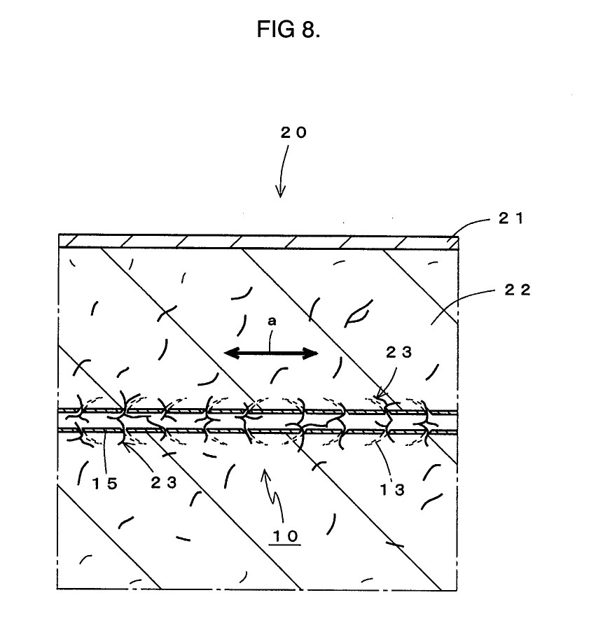

A needle 30 in which the surgical thread for plastic surgery 10 is threaded is inserted from an entry point H to an exit point I in the skin 20 of the face F. The needle 30 is then pulled out, whereby the thread 10 is implanted in the inside skin layers so as to impart a tensing action (arrow a direction) to the skin to eliminate skin sagging 29 (see FIG. 7). Note that in FIG. 7 and FIG. 8, reference numeral 21 indicates the epidermis of the skin. After the thread 10 is implanted in the inside skin layers 22, until the thread body 12 comprised of the absorbable thread is absorbed in the body, the anchoring force of the projections 13 formed at the thread body 12 enables the skin 20 to be given a tensing action in the arrow a direction.

After this, the thread body 12 and projections 13 forming the surgical thread for plastic surgery 10 gradually break down and are absorbed in the inside skin layers 22 and disappear. Note that in the embodiments, not only the thread body 12, but also the projections 13 are made of absorbable thread, while the residual film parts 15 are formed at the parts of the thread body 17 other than the projections (see FIG. 7). The time for the thread body 12 and projections 13 to almost completely break down and be absorbed in the inside skin layers 22 differs depending on the type of the absorbable thread, but usually is about six months. When the thread body 12 and projections 13 break down and are absorbed in the inside skin layers, as shown in FIG. 3, only the cylindrical residual film parts 15 remain in the inside skin layers.

On the other hand, as will be understood from FIG. 8, when the thread 10 is implanted in the inside skin layers 22, new skin tissue 23 is created in the inside skin layers 22. Further, when the residual film part 15 is made from gold, the creation of collagen, one type of skin tissue 23, is further promoted and the skin can be given pliability.

In this way, the thread body 12 and projections 13 gradually break down and are absorbed and new collagen skin tissue 23 is formed in a net fashion around the surgical thread for plastic surgery 10 and from the openings of the residual cylindrical film parts 15. Therefore, the thread and film parts are held by the skin tissue 23 and a tensing action to the skin 20 in the arrow a direction is maintained.

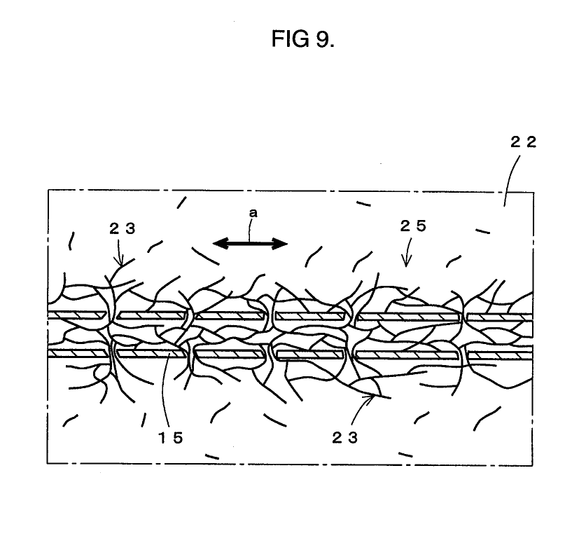

Further, as shown in FIG. 9, after the thread body 12 is absorbed, the skin tissue 23 latching on to the residual film parts 15 forms thread-like parts 25 at the inside skin layers 22, whereby the tensing action given to the skin is maintained. The newly created skin tissue 23 grows in a net fashion entering the cylindrical residual film parts 15 and latching on to the residual film parts 15 whereby the skin tissue 23 forms the thread-like parts 25. The thread-like parts 25 are comprised of the skin tissue 23 naturally created in the inside skin layers 22 of the person whose skin is given the tensing action, so will not cause any allergic reactions etc. and consequently are extremely safe. In addition, unlike with implantation of conventional surgical thread, there is no deterioration along with time, so the skin can be given a tensing action semipermanently.

While the invention has been described with reference to specific embodiments chosen for purpose of illustration, it should be apparent that numerous modifications could be made thereto by those skilled in the art without departing from the basic concept and scope of the invention. For example, the surgical thread for plastic surgery and method of imparting tension to the skin of the present invention are not limited just to the above embodiments. They may also be applied to other locations besides the face.

1. A surgical thread for plastic surgery comprised of a thread shaped member comprised of a thread body, to be implanted in the inside layers of the skin, partially formed in its longitudinal direction with projections for anchoring in the inside skin layers,
wherein at least said thread body is comprised of an absorbable thread, and
said thread body or said projections are formed with residual film parts which will not be absorbed by the inside skin layers.

2. A surgical thread for plastic surgery as set forth in claim 1, wherein said projections are formed integrally with the thread body.

3. A surgical thread for plastic surgery as set forth in claim 1, wherein said projections are thorn shaped members.

4. A surgical thread for plastic surgery as set forth in claim 1, wherein said residual film parts are formed at parts of the thread body other than at said projections.

5. A surgical thread for plastic surgery as set forth in claim 1, wherein the residual film parts are comprised of gold.

6. A method of imparting tension to skin comprising
embedding a surgical thread for plastic surgery as set forth in claim 1 into the inside layers of the skin,
using the anchoring force of the projections of said surgical thread to impart a tensing action to the skin until the thread body comprised of the absorbable thread is absorbed, and
using thread-like parts formed at the inside skin layers by skin tissue latching onto the residual film parts to impart a tensing action after the thread body is absorbed.
